(19) United States
(12) Patent Application Publication
(10) Pub. No.: US 2014/0172103 A1
(43) Pub. Date: Jun. 19, 2014

(54) Polyaxial Intervertebral Cage

(71) Applicants: Michael J. O'Neil, West Barnstable, MA (US); Sheryl Frank, Lakeville, MA (US)

(72) Inventors: Michael J. O'Neil, West Barnstable, MA (US); Sheryl Frank, Lakeville, MA (US)

(21) Appl. No.: 13/716,793

(22) Filed: Dec. 17, 2012

(51) Int. Cl. A61F 2/44; A61F 2/46

(52) U.S. Cl. CPC A61F 2/4455; A61F 2/4611
USPC 623/17.16

(57) ABSTRACT

An intervertebral fusion cage having a first polyaxial joint portion adapted to connect to a corresponding polyaxial joint portion on an inserter so as to form an insertion assembly adapted for polyaxial movement. This assembly allows the cage to be laterally inserted into the problematic lower disc spaces of the spine in a manner such that the cage is desirably substantially parallel to the vertebral endplates upon entry.

### BACKGROUND OF THE INVENTION

Varying access trajectories and approaches are utilized to deliver and implant interbody fusion cages in the lumbar spine. In comparison to conventional anterior or posterior approaches to the lumbar spine, the lateral approach has been shown to minimize posterior and/or anterior tissue/vessel damage as well as reduce operating time, associated blood loss and infection risk. Accordingly, a lateral access approach is frequently selected to deliver interbody fusion cages to the lumbar spine. The lateral approach is generally considered to include the following approaches:
Direct Lateral (transposas entry and parallel cranially and caudally with the disc space),
Anterior-Lateral (anterior to the posas and parallel to the disc space), and
Superior/Inferior lateral (lateral access achieved without parallel entry to the disc space).

Currently marketed cages and instruments typically allow for straight, parallel insertion from a lateral approach. However, one challenge associated with the lateral approach is that the pelvic crest may obstruct a direct lateral approach to the lower intervertebral disc spaces, thereby necessitating a vertically-angled approach to these lower disc spaces. This challenge is particularly pertinent for approach to the L5/S1 disc space. As a result, a cage so inserted may undesirably enter the disc space at an angle and may permanently remain at that angle. As a result, some systems allow for angulations and control in the axial plane.

This desire to provide flexibility for multiple lateral approaches based upon patient indications promotes the need for varying implant, attachment features and/or insertion instruments for each approach increasing overall procedure complexity and cost.

US2011-0125266 (Nuvasive) discloses a three-piece cage having central articulation. Nuvasive also discloses a flexible cage.

WO2010-075555 (Spann) discloses a one-piece vertically articulating cage having proximal articulation.

US2011-0029083 (Medtronic) discloses a multi-piece cage having central articulation.

US2011-0320000 (DePuy Spine I) discloses a multi-piece cage with central articulation.

US2011-0319998 (DePuy Spine II) discloses a polyaxial trial.

In addition, conventional cages have been damaged or fractured during insertion, impaction or manipulation in the disc space. This is partially due to the fact that insertion features are typically placed on the surface of or within the proximal wall of the cage, thereby creating stress risers and reducing overall cage resistance to breakage.

### SUMMARY OF THE INVENTION

The present invention relates to an intervertebral fusion cage having a first polyaxial joint portion adapted to connect to a corresponding polyaxial joint portion on an inserter so as to form an insertion assembly adapted for polyaxial movement. This assembly allows the cage to be laterally inserted into the problematic lower disc spaces of the spine in a manner such that the cage is desirably substantially parallel to the vertebral endplates upon entry.

The disclosed polyaxial cage implants and instruments allow for flexible and adaptable angles of approach based upon patient anatomy, surgical preference and numbers of levels to be fused. This ability to control angle enables multilevel procedures through a single port and can also enable access to L5/S1.

Therefore, in accordance with the present invention, there is provided an intervertebral fusion cage, comprising:
a) an anterior wall, a posterior wall, a proximal sidewall and a distal sidewall, the sidewalls connecting the anterior wall and the posterior wall to form a throughhole,
b) an upper surface and a lower surface, each surface adapted for gripping a respective adjacent vertebral endplate,
c) a first joint portion extending from the proximal sidewall and adapted to provide polyaxial movement of the cage.

Also in accordance with the present invention, there is provided an assembly comprising:
i) intervertebral fusion cage, comprising:
   a) an anterior wall, a posterior wall, a proximal sidewall and a distal sidewall, the sidewalls connecting the anterior wall and the posterior wall to form a throughhole,
   b) an upper surface and a lower surface, each surface adapted for gripping a respective adjacent vertebral endplate,
   c) a first joint portion extending from the proximal sidewall and adapted to provide polyaxial movement of the cage, and
ii) an inserter having a proximal handle and a distal end portion, wherein the distal end portion comprises a second joint portion,
wherein the first and second joint portion form a polyaxial joint.

Also in accordance with the present invention, there is provided a method comprising the steps of:
a) providing the assembly of the present invention,
b) inserting the cage of the assembly into a disc space via a lateral approach.

### DESCRIPTION OF THE FIGURES

FIGS. 1a-b disclose embodiments of the polyaxial cage of the present invention.

FIGS. 2a-2b show implantation of a cage of the present invention via an antero-lateral approach.

FIGS. 3a-3b show implantation of a cage of the present invention with cranial-caudal angulation.

FIGS. 4a-4c show implantation of a cage of the present invention with cranial-caudal and antero-lateral angulations.

FIGS. 5a-b disclose cross-section of a ball-type polyaxial cage of the present invention in unlocked and locked modes with an inserter.

FIGS. 6a-b disclose side views of a ball-type polyaxial cage of the present invention in unlocked and locked modes with an inserter.

FIG. 7 discloses a side view of an implanted cage of the present invention.

FIGS. 8a-b disclose a cage of the present invention having a modular joint portion.

FIGS. 9a-b disclose a cage of the present invention having a break-away joint portion.

FIGS. 10a-c disclose cross-sections of a recess-type polyaxial cage of the present invention in unlocked modes with an inserter.

FIGS. 11-12a discloses a push version of a recess-type polyaxial cage of the present invention in unlocked mode with an inserter.

FIG. 12b discloses a push version of a recess-type polyaxial cage of the present invention in locked mode with an inserter.

FIGS. 13a-b disclose a recess-type polyaxial cage of the present invention in locked and unlocked modes with an inserter.

FIGS. 14a-b disclose a pull version of a recess-type polyaxial cage of the present invention in locked and unlocked modes with an inserter.

FIG. 15 disclose a polyaxial cage of the present invention used in conjunction with a steering finger.

FIGS. 16a-e disclose a steerable polyaxial joint of the present invention.

FIGS. 17a-i disclose means for measuring the angle of the implant.

### DETAILED DESCRIPTION OF THE INVENTION

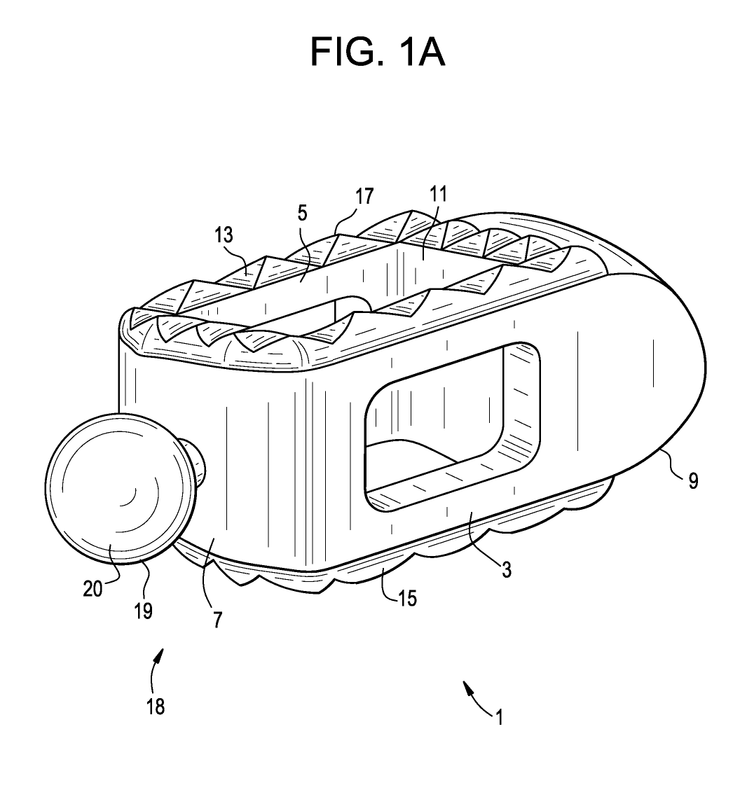

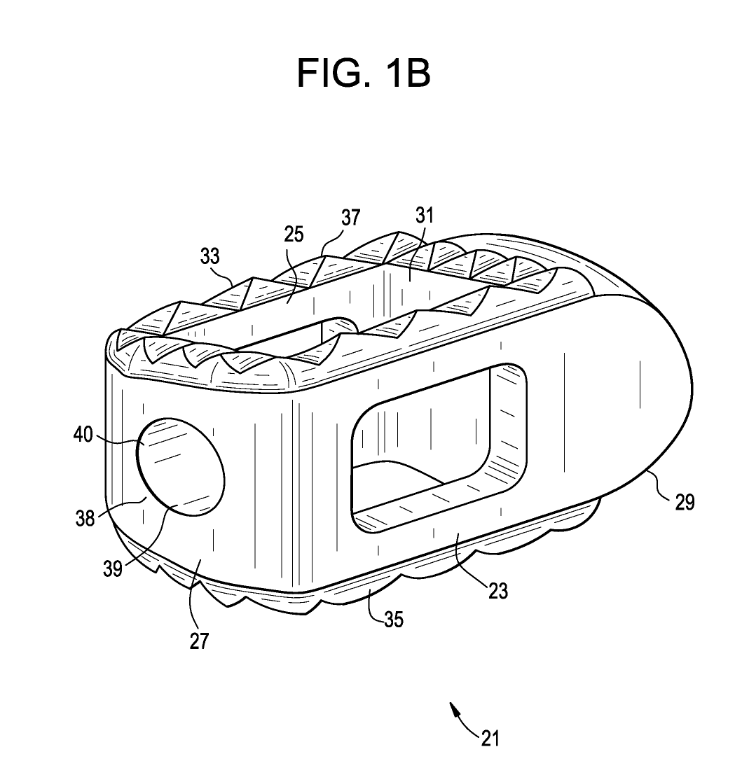

In some embodiments, it is contemplated that the polyaxial cages has a ball-type joint portion. The ball portion of this cage is meant to polyaxially articulate with a corresponding spherical recess provided on an inserter. Now referring to FIG. 1a, there is provided an intervertebral fusion cage 1, comprising:
a) an anterior wall 3, a posterior wall 5, a proximal sidewall 7 and a distal sidewall 9, the sidewalls connecting the anterior wall and the posterior wall to form a throughhole 11,
b) an upper surface 13 and a lower surface 15, each surface having teeth 17 adapted for gripping a respective adjacent vertebral endplate,
c) a first joint portion 18 extending from the proximal sidewall and adapted to provide polyaxial movement of the cage.
   wherein the first joint portion comprises a ball 19 having a portion of a sphere 20.

In some embodiments, it is contemplated that the polyaxial cages has a spherical recess-type joint portion. The spherical recess portion of this cage is meant to polyaxially articulate with a corresponding ball portion provided on an inserter. Now referring to FIG. 1b, there is provided an intervertebral fusion cage 21, comprising:
a) an anterior wall 23, a posterior wall 25, a proximal sidewall 27 and a distal sidewall 29, the sidewalls connecting the anterior wall and the posterior wall to form a throughhole 31,
b) an upper surface 33 and a lower surface 35, each surface having teeth 37 adapted for gripping a respective adjacent vertebral endplate,
c) a first joint portion 38 extending from the proximal sidewall and adapted to provide polyaxial movement of the cage,
   wherein the joint portion comprises a recess 39 having a substantially hemispherical portion 40.

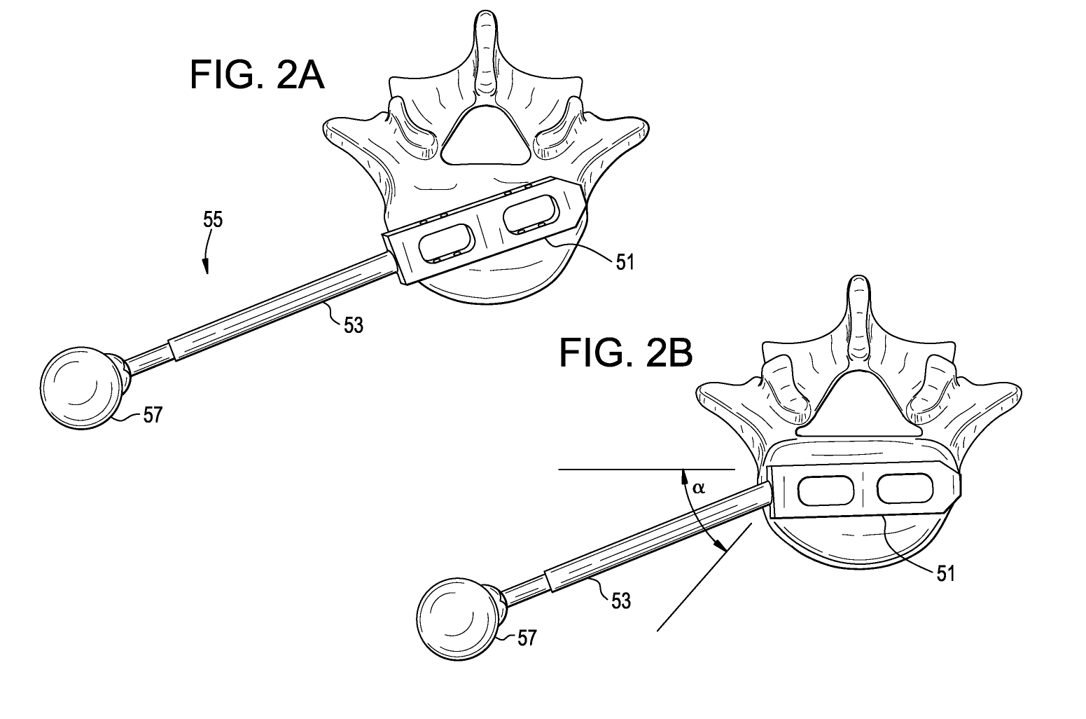

Now referring to FIGS. 2-4, cages with polyaxial means for insertion via various anterior/posterior and/or cranial/caudal trajectories include polyaxial features on the proximal surface of the cage for securement and intra-discal insertion and adjustment in various planes.

For example, in FIGS. 2a-2b, there are provided examples of a cage of the present invention that has been inserted into the disc space by an antero-lateral approach using an assembly of the present invention. The initial insertion into the disc space is carried out with the cage 51 being substantially co-linear with the shaft 53 of the inserter 55, as in FIG. 2a. After the cage has been inserted, and now referring to FIG. 2b, the handle 55 of the inserter is manipulated so as to angulate the cage and bring it to an orientation that is more orthogonal to the anterior-posterior axis. As shown, the assembly of the present invention can provide such angulation in an amount of up to an angle α at least 60 degrees.

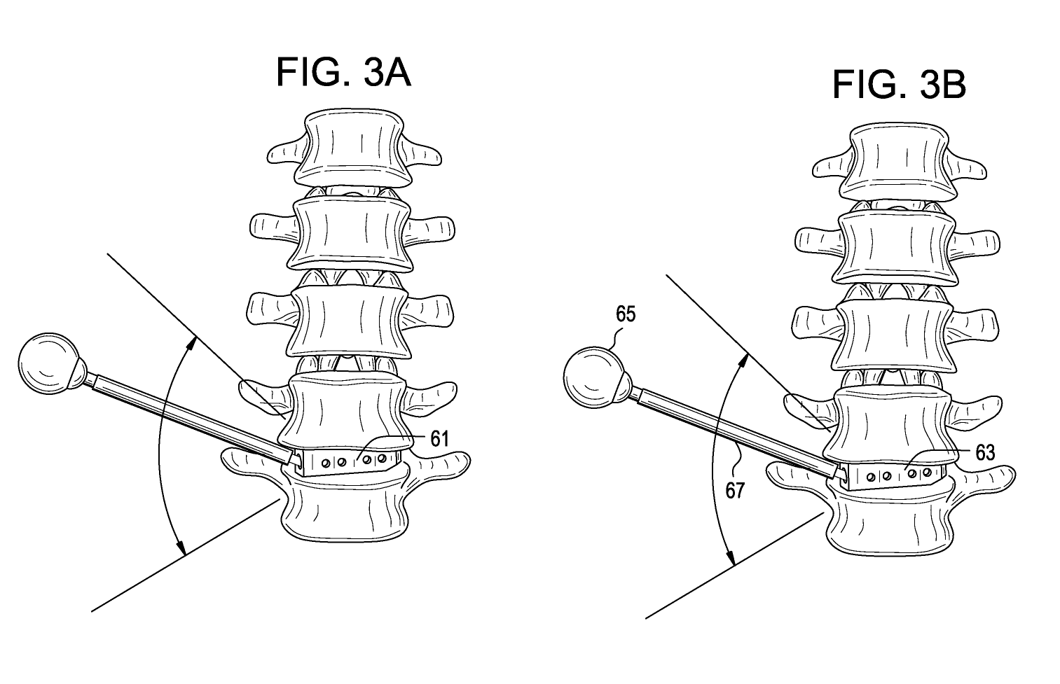

FIGS. 3a-b disclose how the polyaxial cage-inserter assembly may be used when inserting a cage above or below the level of the incision. FIG. 3a disclose how the polyaxial joint allows the cage 61 to be inserted in a manner parallel to the endplate of the adjacent vertebral bodies, thereby providing for desirable parallelism within the disc space. FIG. 3b discloses how the same incision can be used as a starting point for the insertion of cages of three different levels. In this FIG., the surgeon inserts the first cage 63 and then can withdraw the handle 65 and shaft 67 so as to attach another cage (not shown). The incision provided for the first cage can then be used as an entry point for the placement of the second cage, and so on.

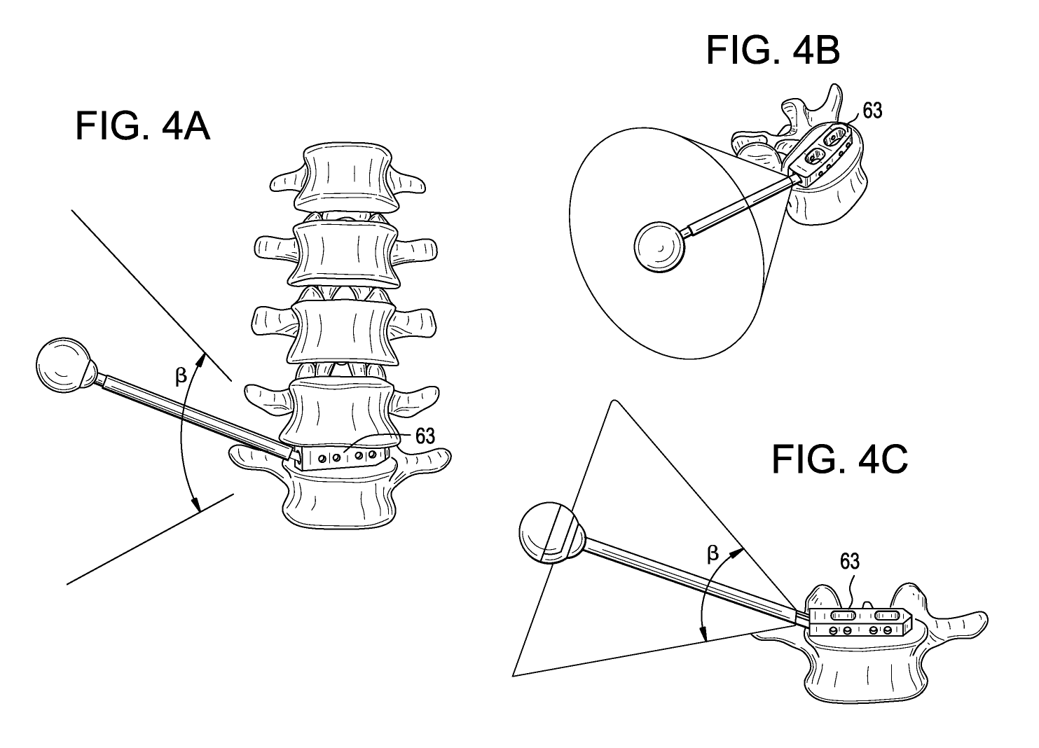

FIGS. 4a-c disclose the real-life situation in which there is deviation from the purely lateral approach in both the anterior-posterior and cranial-caudal directions. In this case, the assembly provides for an angulation cone of access having an angle β of up to about 60 degrees.

The polyaxial articulating means allows the surgeon to insert an implant through an MIS approach at various access trajectories and to various levels. Typically, the polyaxial joint can provide a cone of access in angles of up to about 60 degrees in both the anterior-posterior and cranial-caudal directions.

The polyaxial cage can be adjusted intra-operatively (on the mayo stand) or in-situ (during or following insertion into the disc). When performed in-situ, the cage can be securely held for initial insertion and impaction, or loosened as desired for steering upon advancement into the disc space.

In one implantation embodiment, the cage implant is initially securely held and partially inserted into the prepared disc space. The user then loosens the knob to reduce secure holding of the implant and then adjusts the handle on the implant to a different angle. Alternatively, the surgeon may leave the handle loose and continue to impact, thereby allowing the implant to freely turn as it enters the disc space. This configuration is especially advantageous to the TLIF approach (or other surgical approaches) as it would be able to angle in multiple planes to avoid anatomy that may be in the way of a direct approach to the disc space. Once the implant is in place, the handle can be removed, leaving behind the cage at the desired location.

One preferred embodiment of the invention is an intervertebral implant with a polyaxial feature (recessed or protruding) that allows for pivoting in a trajectory that is generally conical to the access point into the disc. The pivoting feature is universal in nature and utilizes a recessed or protruding ball-and-socket joint that allows the articulation to occur in multiple planes, creating a cone of access.

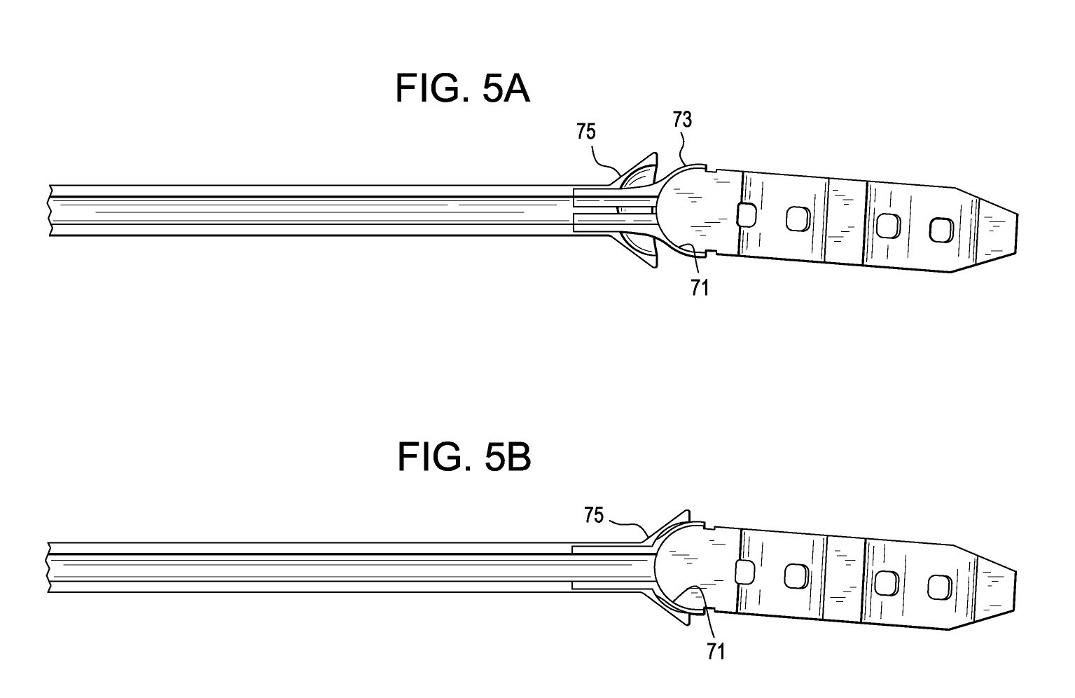

Now referring to FIGS. 5-7, in protruding ball polyaxial cage embodiments, the cage incorporates a protruding polyaxial ball feature 71 that is generally spherical in shape. A compression collet 73 affixes to the protruding polyaxial feature by press fit means. Once attached, the pusher 75 is advanced via manual compression, threading, ratcheting means, or other means to increase the grip or hold onto the cage. Advancement of the pusher upon the collet compresses on the proximal side of the collet, which subsequently compresses onto the protruding polyaxial feature for cage securement.

In some embodiments, the ball of the cage is substantially hemispherical, as in FIG. 5. In some embodiments, the substantially hemispherical ball is formed in the proximal sidewall (as in FIG. 5). These ball-type cages of the present invention so formed allow for inserter attachment to the outside of the cage, thereby reducing stress risers, maximizing wall thickness, and reducing the likelihood of breakage.

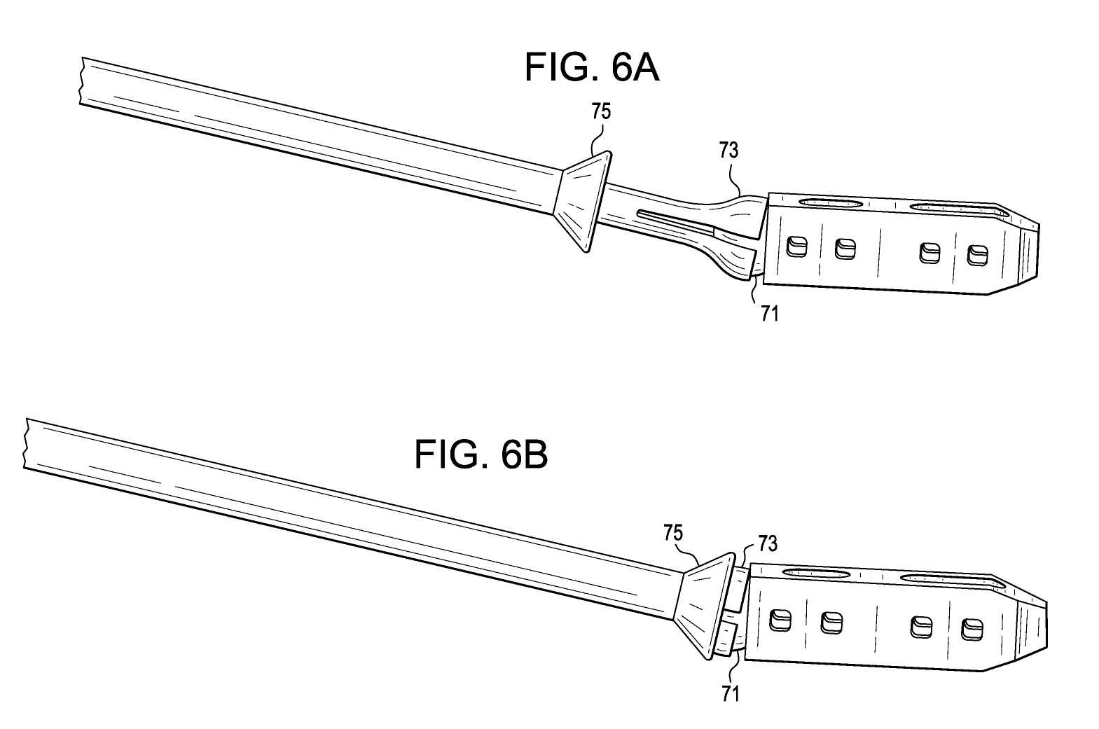

FIGS. 6a-6b disclose how the cage and inserter are attached so as to lock the cage at a fixed angle. In FIG. 6a, the collet 73 contacts the ball 71 but is not lockled in place. Distal movement of the pusher (as shown by arrows in FIG. 6a) produces a locked assembly (as shown in FIG. 6b).

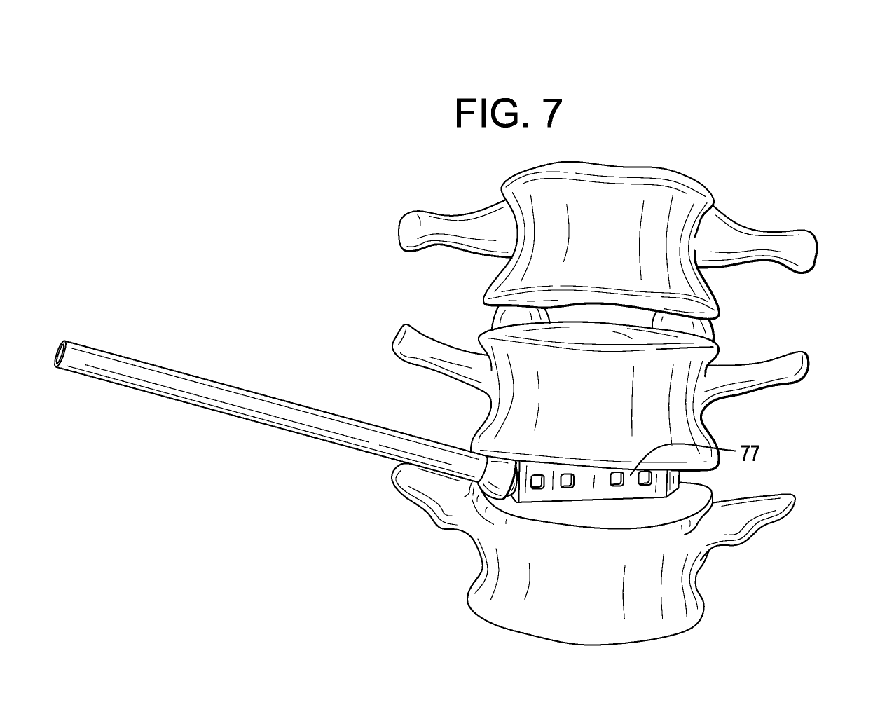

FIG. 7 shows the cage 77 implanted in the disc space through an angled approach.

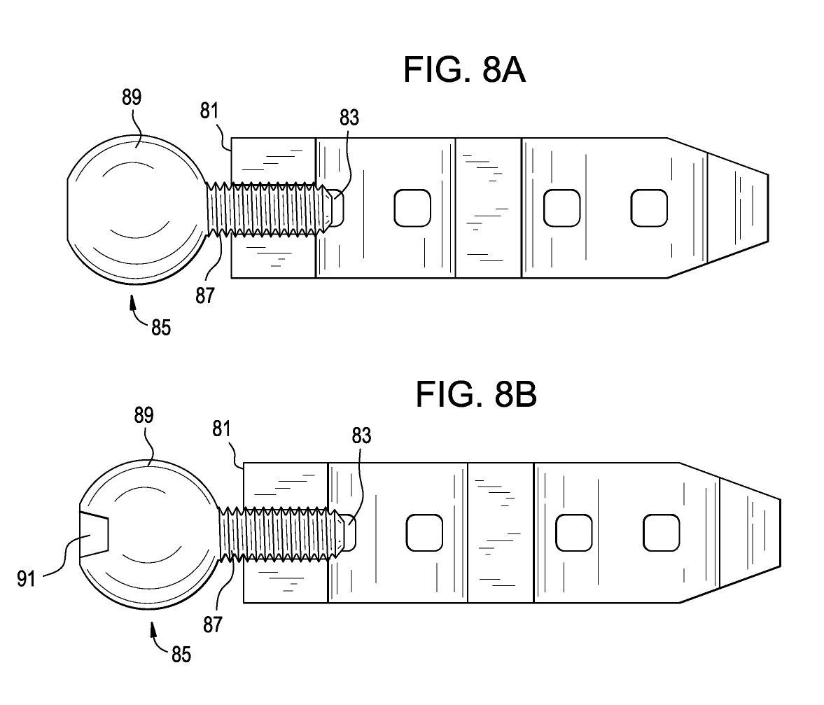

Now referring to FIGS. 8a-b, the protruding polyaxial ball-type feature can be modular and may be optionally removed following implantation. The feature can be attached by any conventional means. For example, a threadably-attached embodiment of modularity is shown in FIGS. 8a. Thus, in some embodiments, the proximal sidewall 81 comprises a threaded recess 83, the joint portion 85 further comprises a threaded pin 87 extending from the ball 89, and the threaded pin is threadably received in the threaded recess. In some embodiments thereof, the ball is substantially spherical. Although shown with threaded means, the modular attachment can be any typically modular attachment, including a press fit, or a keyway attachment.

FIG. 8b discloses the same modular attachment, but with a drive feature 91 added to the ball. This drive feature may be used to insert the joint portion 85 into the remainder of the cage, and to remove it therefrom as well.

In some embodiments, the joint portion is metallic (for extra strength) while the proximal sidewall comprises a polymer (so that its elasticity is more compatible with bone). In other higher-strength embodiments, wherein the joint portion is metallic, the proximal sidewall of the cage is metallic (for extra strength), and the distal wall of the cage comprises a polymer.

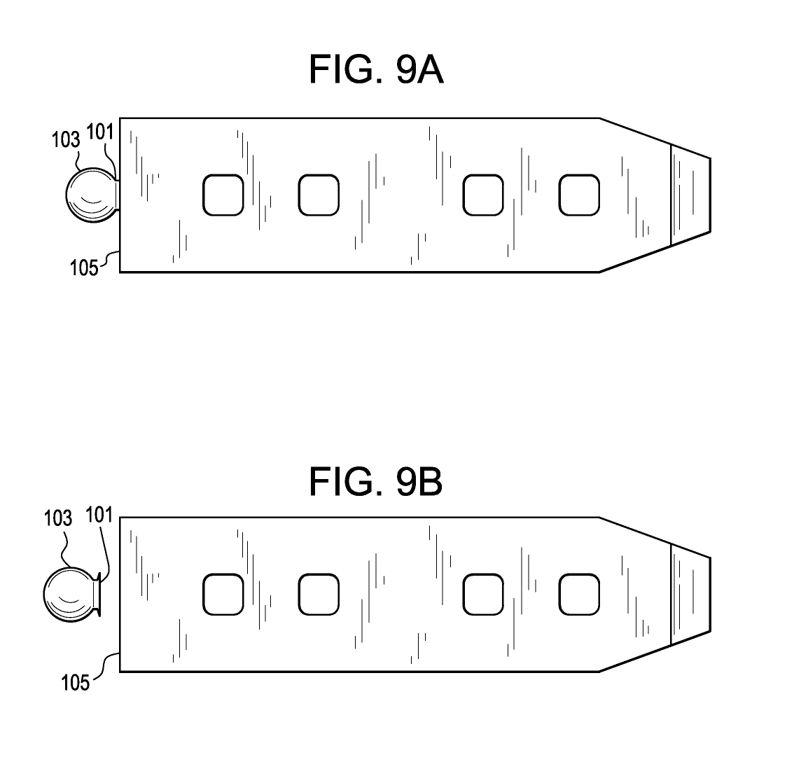

Now referring to FIGS. 9a-b, the protruding polyaxial ball-type feature can be a break-away type and may optionally be removed following implantation. Thus, in some embodiments, as in FIG. 9a, the joint portion further comprises a flange 101, wherein the flange attaches to the ball 103 and to the proximal sidewall 105. Preferably, as in FIG. 9b, the flange is adapted for breakage by manual force. This break-away flange allows the ball to be removed from the cage once the joint portion has performed its polyaxial function, thereby reducing the profile of the cage.

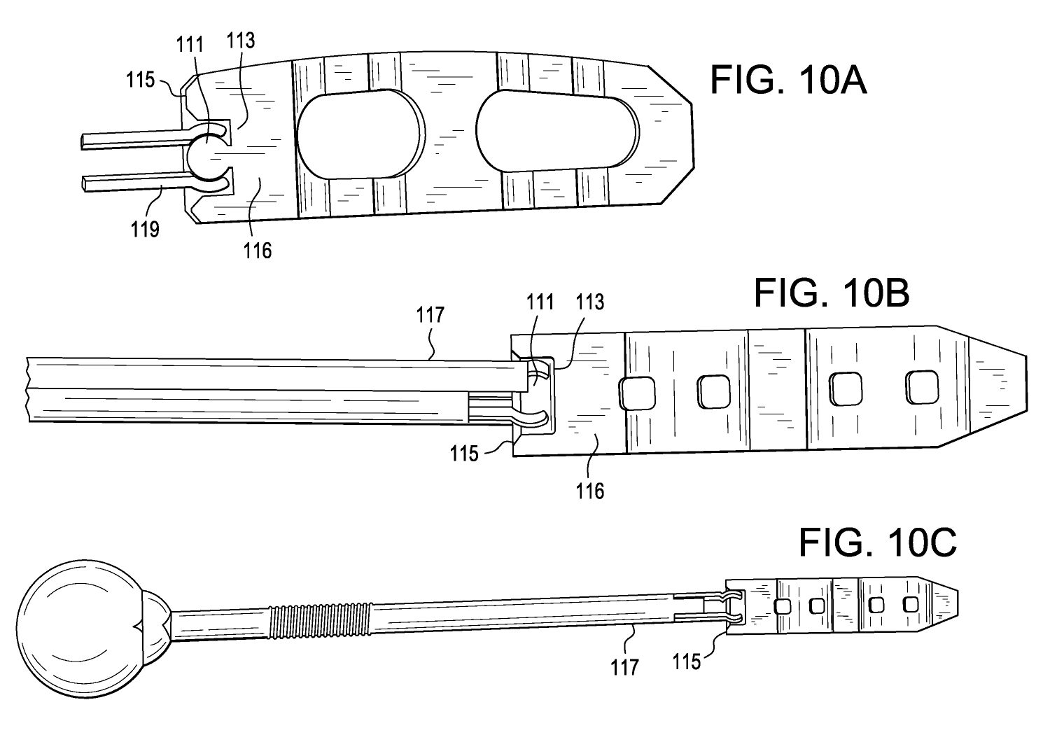

Now referring to FIGS. 10a-c, the protruding ball-type polyaxial feature can also be recessed beneath the proximal surface of the proximal wall of the cage to allow for a larger contact area upon the vertebral body. Thus, in some embodiments, the ball-type joint portion 111 is substantially formed within a recess 113 in the proximal surface 115 of the proximal sidewall 116, as in FIG. 10a. A cannulated pusher 117 and internal compression collet 119 are employed to secure to the cage, as in FIGS. 10b-c.

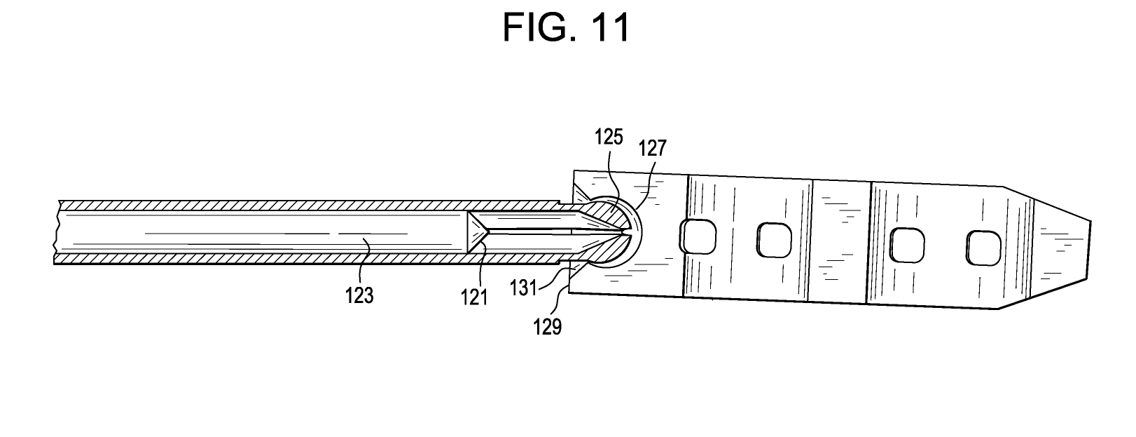

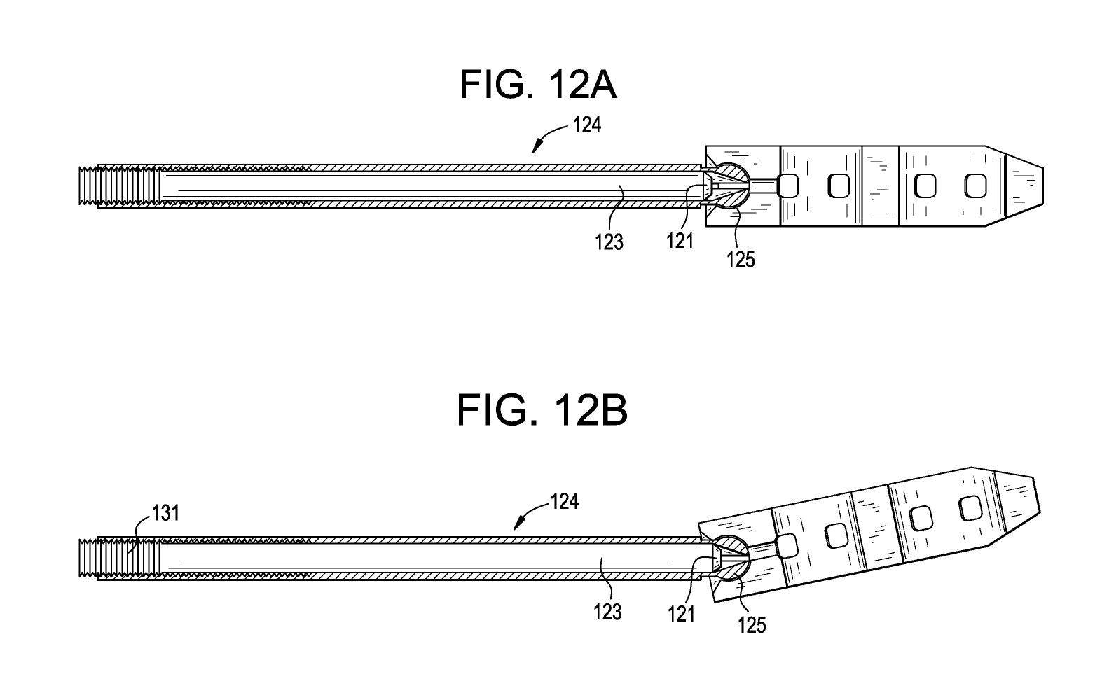

In some embodiments, the polyaxial feature can be a recessed feature that is generally spherical in shape, thereby enabling the cage to pivot in an access trajectory that is generally conical. In some embodiments, and now referring to FIGS. 11-12b, the joint portion comprises a recess 127 having a substantially hemispherical portion, wherein the recess is formed in the proximal sidewall 129. Preferably, the recess further comprises a chamfer 131 extending proximally from substantially hemispherical portion. A cannulated expansion collet 125 is inserted into the recessed polyaxial feature, a pusher 123 is then advanced through the cannulated portion of the collet to expand the collet and secure the inserter to the cage at the desired anterior/posterior and/or cranial/caudal angles. FIGS. 11-12a show the assembly in its unlocked form, wherein the distal end 121 of the pusher 123 is within the cannulated proximal portion 124 of the collet tool, but not engaged with the collet head 125. FIG. 12a shows the assembly in its locked form, wherein the distal end 121 of the pusher 123 is engaged with the collet head 125. FIG. 12b also discloses that the proximal end portion of the pusher may comprise an advancement means, such as a thread 131.

In some of the expanding collet concepts disclosed herein, the wall thickness of the cage has been reduced. However, it is contemplated that the attachment force for the assembly is controllable and can be reduced to allow for cage slippage in the recesses polyaxial feature prior to breakage.

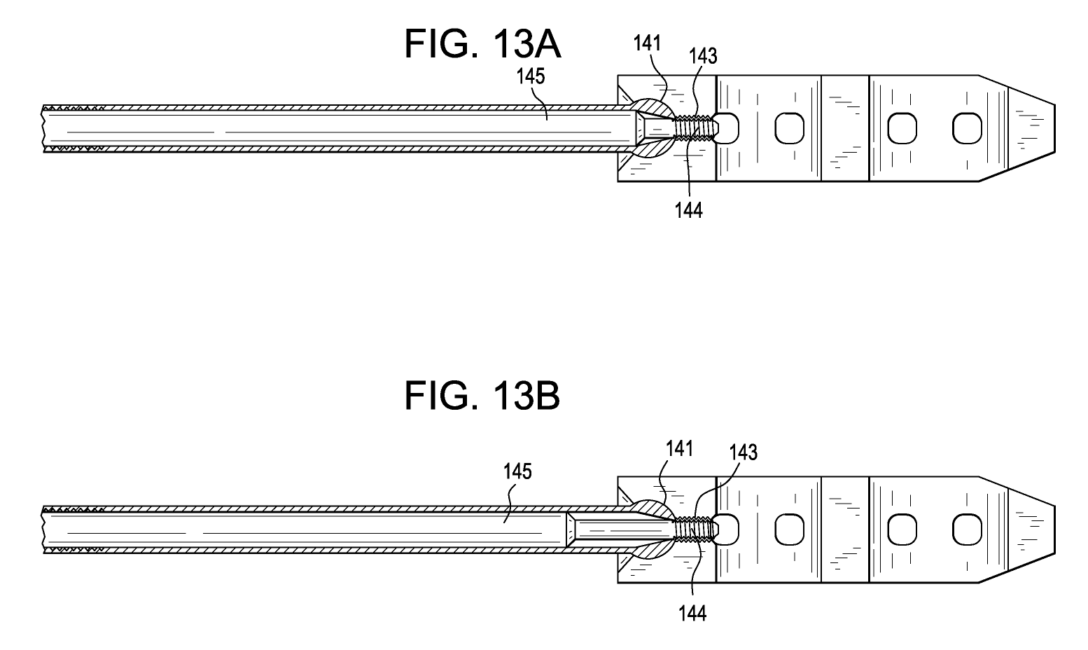

Now referring to FIGS. 13a-b, an additional inner securement mechanism can be employed to augment the securement strength of the assembly and allow for secure attachment if extraction or revision is required. This mechanism can be a threaded wire 144 provided at the distal end of the pusher 145, as is shown in FIG. 13a-b. This threaded wire can be received in a corresponding threaded portion of the recess. This threaded through hole can also be used to backfill graft into the cage as well. Thus, in some embodiments, the polyaxial recess of the cage further includes a threaded portion 143 extending distally from the substantially hemispherical portion 141 of the recess.

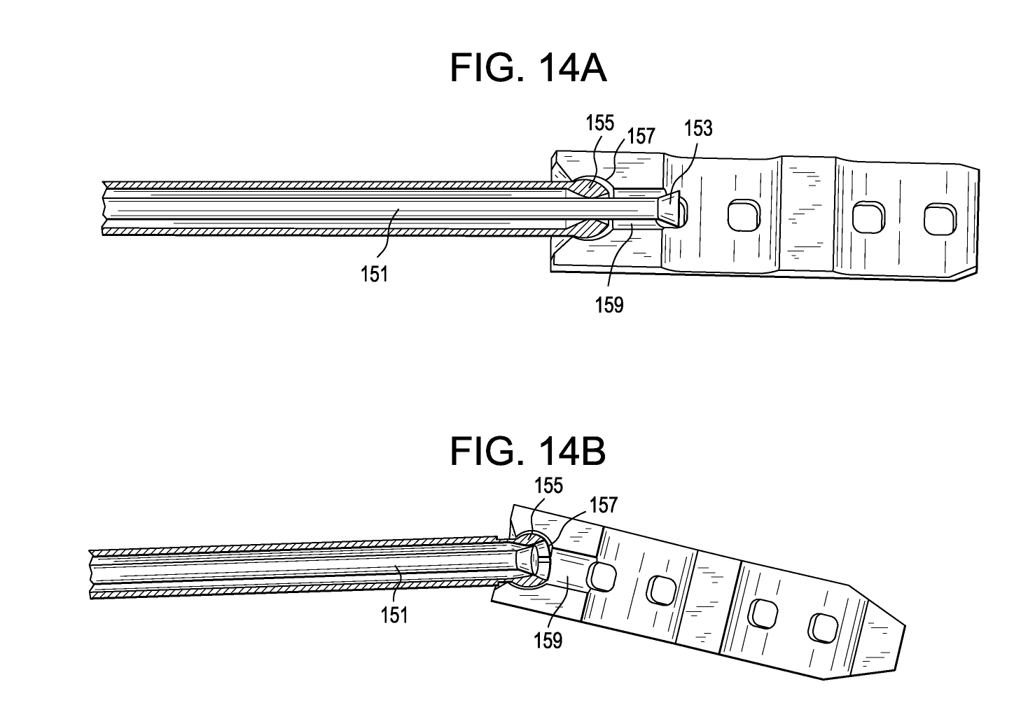

Now referring to FIG. 14, inserter attachment to the inner polyaxial recess feature (which is generally spherical in shape) can also be accomplished with a “pull” mechanism. A central puller 151 with a tapered distal head 153 is withdrawn to expand collet 155 into the recessed polyaxial feature 157, thereby locking the assembly. Thus, in some embodiments, the recess further includes a channel 159 extending distally from the substantially hemispherical portion.

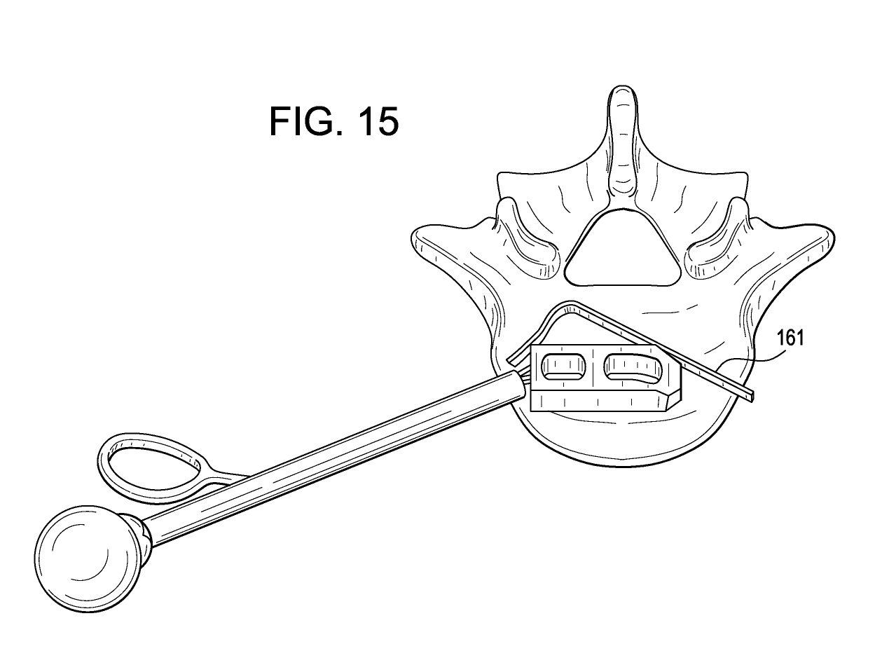

Now referring to FIG. 15, in some embodiments, a steerable finger 161 or blade is inserted into the disc space first and actuated into the desired direction. This finger assists in steering or controlling the implant to the desired location as insertion force is applied. The finger is removed during or after placement. The finger can be attached to the polyaxial inserter as is seen in FIG. 15. The finger can be comprised of a memory metal blade which allows for angulations as deployed from the inserter.

Although all embodiments allow for implantation at various trajectories, one preferred embodiment is the outer polyaxial system shown in FIGS. 5a and 5b (due to enhanced device strength) with the steerable finger as shown in FIG. 15 to enhance steering capabilities for challenging trajectories.

The same polyaxial features can be employed upon modular articulating instruments, allowing for interchangeable heads or working ends for use at various access trajectories, as disclosed in the US provisional patent application, filed even date, entitled “Polyaxial Articulating Tool”, Frasier et al., (Attorney Docket No. DEP6625USSP) the specification of which is incorporated by reference in its entirety

The polyaxial features of the present invention can have a generally smooth surface to allow for unconstrained/infinite adjustment or can have features which allow for adjustment to specific desired angulations. These features can include undercut, rings, spikes, teeth or facets.

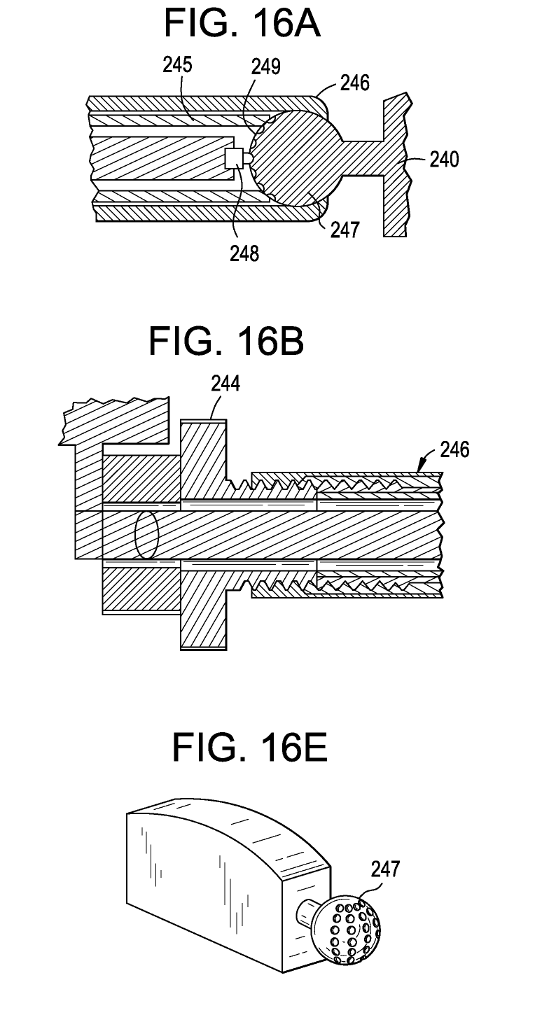

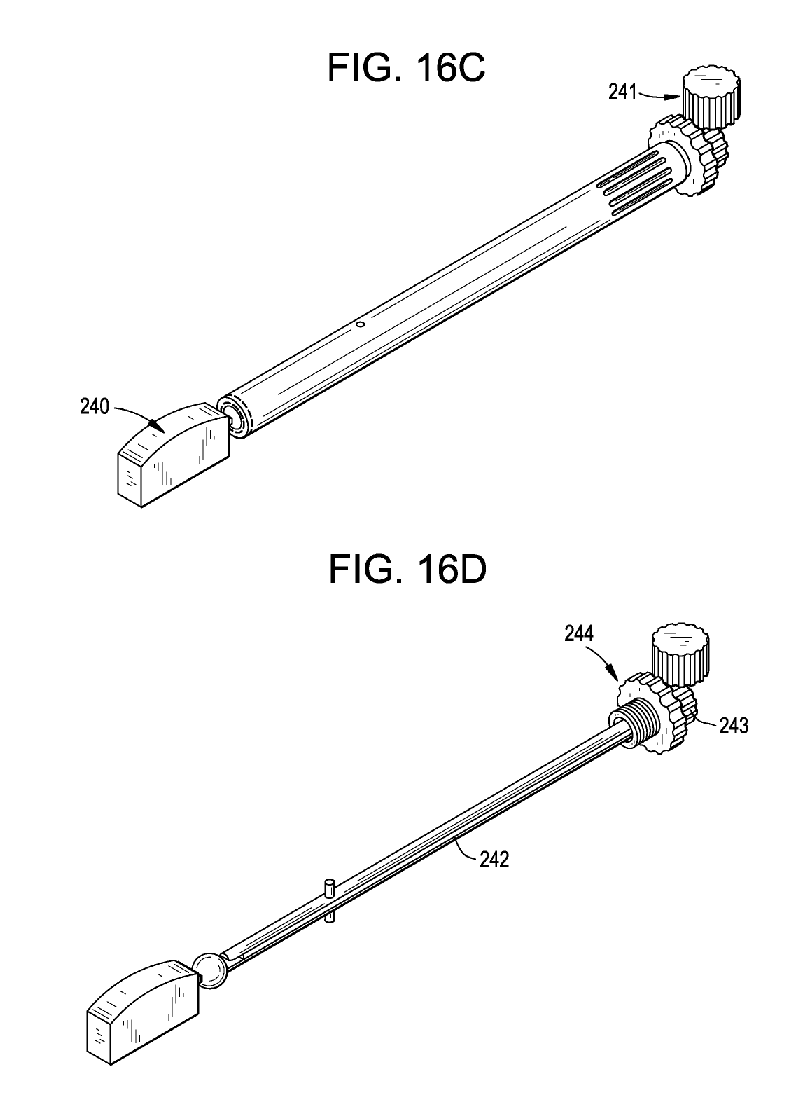

Active embodiments of angle adjustment allow for remote angle adjustment or steering. Steering can be accomplished with either a tension cable, a pusher member as is known in the art, or with a belt drive means as is shown in (FIGS. 16a-16e). The belt drive embodiment has an internal shaft with a continuous toothed belt 242, much like a conveyer belt or chain saw bar and chain. The proximal end of the instrument has three knobs. One knob 241 drives the belt 242, another 243 changes the plane in which the belt resides by turning it about the long axis of the instrument. The third knob 244 tightens the inner 245 and outer 246 sleeves against the spherical end 247 of the implant, locking its position. The protrusions 248 on the belt interface with mating features 249 on the spherical end of the implant 240. Driving the belt causes the implant 240 angle to change in one direction. Changing the angle of the drive belt 242 allows adjustment of the implant angle in a second plane. Through a combination of driving the belt with knob 241 and turning the belt drive with knob 243 around the long axis of the instrument an infinite number of combinations of angles and positions of the implant can be achieved. Once the preferred angle is achieved, this can be locked in place by tightening the inner 245 and outer 246 sleeves against the spherical end 247 of the implant. The spherical end of the implant then detaches from the main implant body leaving the implant behind.

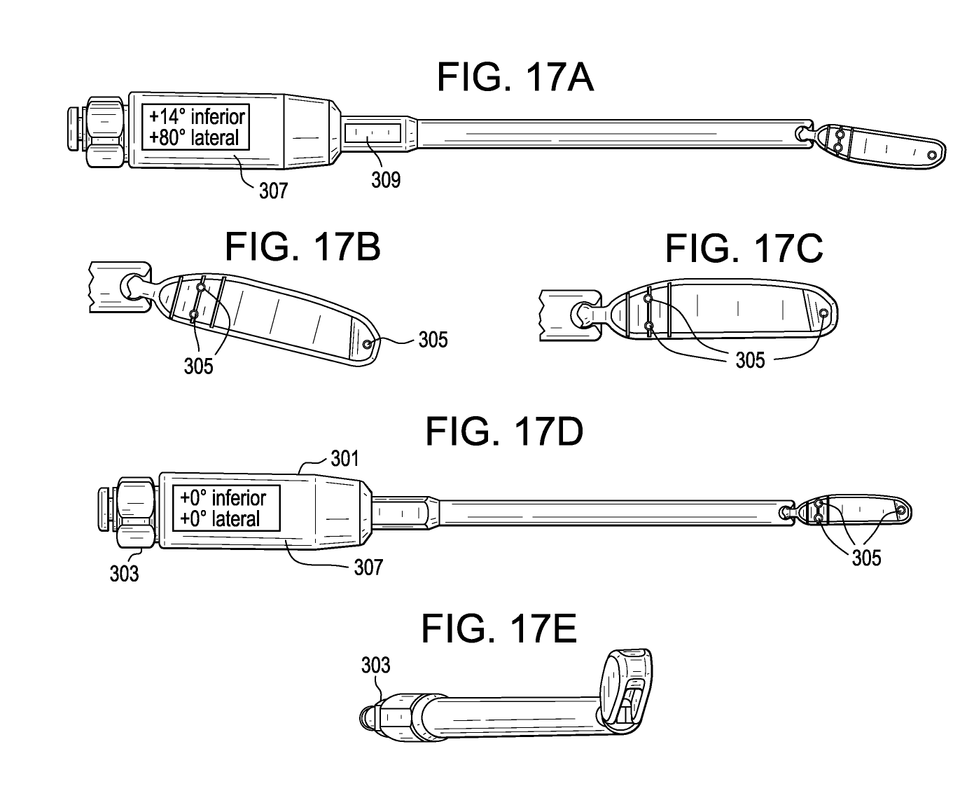

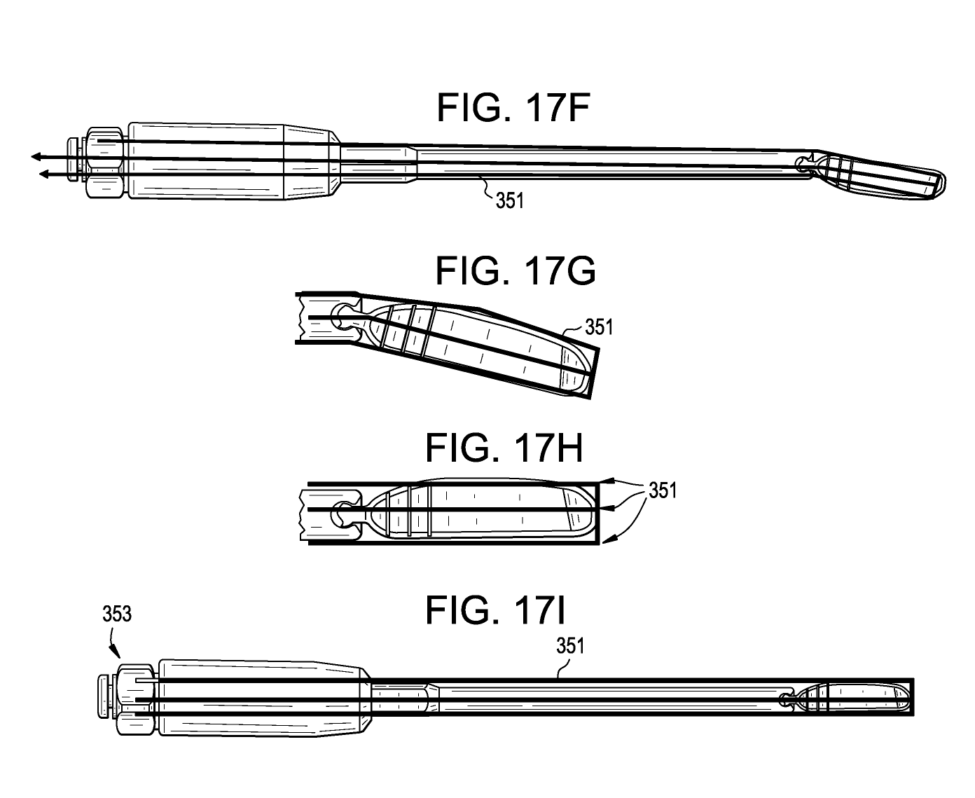

The implant (or articulating trials and inserters) can also provide a means to measure the angulation during articulation. This angle is determined by assessing the difference between the angle following insertion and the angle following articulation. This differential angle is then utilized to ensure the implant is also placed at the identical angle, thereby ensuring trial and implant placement are consistent. Such means are shown in FIGS. 17a-i. In FIGS. 17a-e, the angle can be determined with the use of radiofrequency (RF) triangulation means. Handle 301 contains an RF emitter or reader 303 and three or more passive markers 305 are contained at known locations with the implant/trial(s). Based upon RF tracking of markers, the handle provides a graphic display 307 of trial orientation in both superior/inferior and right/left lateral planes relative to a known reference flat 309 on the handle.

FIG. 17f-i show the use of angle tracking by measuring movement of cables 351 that provide tensioning. Rotation of a knob 353 located on the handle tensions the cable. The displacement of the cable upon the knob provides for calculation of the angle through a sensor (not shown).

In some embodiments, the cage of the present invention comprises a plurality of radiographic markers to help the surgeon fluoroscopically visualize the cage's placement within the disc space. In some embodiments, the cage has three markers—one at each end and one in the central region. In some embodiments, the markers are in bead form, while in others the markers are in wire form.

The cages of the present invention may be made from any material appropriate for human surgical implantation, including but not limited to, surgically appropriate metals, and non-metallic materials, such as carbon fiber composites, polymers, ceramics, and allograft materials.

The interbody devices are preferably made out of PEEK or CFRP or any other suitable material providing adequate strength and radiolucency. However, implantable metals such as titanium or stainless steel components may be required to ensure adequate strength for either the interbody device. In some cases the interbody device can be made as a combination of PEEK and metal. In some cases, resorbable materials such as polylactide, polyglycolide, and magnesium are preferred.

In some embodiments, the cage material is selected from the group consisting of PEEK, ceramic and metallic. The cage material is preferably selected from the group consisting of metal and composite (such as PEEK/carbon fiber).

If a metal is chosen as the material of construction for a component, then the metal is preferably selected from the group consisting of titanium, titanium alloys (such as Ti-6Al-4V), chrome alloys (such as CrCo or Cr—Co—Mo) and stainless steel.

If a polymer is chosen as a material of construction for a component, then the polymer is preferably selected from the group consisting of polyesters, (particularly aromatic esters such as polyalkylene terephthalates, polyamides; polyalkenes; poly(vinyl fluoride); PTFE; polyarylethyl ketone PAEK; polyphenylene and mixtures thereof.

If a ceramic is chosen as the material of construction for a component, then the ceramic is preferably selected from the group consisting of alumina, zirconia and mixtures thereof It is preferred to select an alumina-zirconia ceramic, such as BIOLOX delta™, available from CeramTec of Plochingen, Germany. Depending on the material chosen, a smooth surface coating may be provided thereon to improve performance and reduce particulate wear debris.

In some embodiments, the cage member comprises PEEK. In others, it is a ceramic.

In some embodiments, the first component consists essentially of a metallic material, preferably a titanium alloy or a chrome-cobalt alloy.

In some embodiments, the components are made of a stainless steel alloy, preferably BioDur® CCM Plus® Alloy available from Carpenter Specialty Alloys, Carpenter Technology Corporation of Wyomissing, Pa. In some embodiments, the outer surfaces of the components are coated with a sintered beadcoating, preferably Porocoat™, available from DePuy Orthopaedics of Warsaw, Ind.

In some embodiments, the components are made from a composite comprising carbon fiber. Composites comprising carbon fiber are advantageous in that they typically have a strength and stiffness that is superior to neat polymer materials such as a polyarylethyl ketone PAEK. In some embodiments, each component is made from a polymer composite such as a PEKK-carbon fiber composite.

Preferably, the composite comprising carbon fiber further comprises a polymer. Preferably, the polymer is a polyarylethyl ketone (PAEK). More preferably, the PAEK is selected from the group consisting of polyetherether ketone (PEEK), polyether ketone ketone (PEKK) and polyether ketone (PEK). In preferred embodiments, the PAEK is PEEK.

In some embodiments, the carbon fiber comprises between 1 vol % and 60 vol % (more preferably, between 10 vol % and 50 vol %) of the composite. In some embodiments, the polymer and carbon fibers are homogeneously mixed. In others, the material is a laminate. In some embodiments, the carbon fiber is present in a chopped state. Preferably, the chopped carbon fibers have a median length of between 1 mm and 12 mm, more preferably between 4.5 mm and 7.5 mm. In some embodiments, the carbon fiber is present as continuous strands.

In especially preferred embodiments, the composite comprises:
−40-99% (more preferably, 60-80 vol %) polyarylethyl ketone (PAEK), and
—1-60% (more preferably, 20-40 vol %) carbon fiber,
wherein the polyarylethyl ketone (PAEK) is selected from the group consisting of polyetherether ketone (PEEK), polyether ketone ketone (PEKK) and polyether ketone (PEK).

In some embodiments, the composite consists essentially of PAEK and carbon fiber. More preferably, the composite comprises 60-80 wt % PAEK and 20-40 wt % carbon fiber. Still more preferably the composite comprises 65-75 wt % PAEK and 25-35 wt % carbon fiber.

In some embodiments using a ball joint portion , the joint portion of the cage is metallic and the proximal sidewall comprises a polymer. In others, the joint portion is metallic, the proximal sidewall is metallic, and the distal wall comprises a polymer.

Although the present invention has been described with reference to its preferred embodiments, those skillful in the art will recognize changes that may be made in form and structure which do not depart from the spirit of the invention.

We claim:

1. An intervertebral fusion cage, comprising:
a) an anterior wall, a posterior wall, a proximal sidewall and a distal sidewall, the sidewalls connecting the anterior wall and the posterior wall to form a throughhole,
b) an upper surface and a lower surface, each surface adapted for gripping a respective adjacent vertebral endplate,
c) a first joint portion extending from the proximal sidewall and adapted to provide polyaxial movement of the cage.

2. The cage of claim 1 wherein the first joint portion comprises a ball having a portion of a sphere.

3. The cage of claim 2 wherein the ball is substantially hemispherical.

4. The cage of claim 3 wherein the substantially hemispherical ball is formed in the proximal sidewall.

5. The cage of claim 2 wherein the proximal sidewall comprises a recess, and the joint portion further comprises a pin extending from the ball, and the pin is received in the recess.

6. The cage of claim 2 wherein the first joint portion is modular.

7. The cage of claim 5 wherein the joint portion is metallic and the proximal sidewall comprises a polymer.

8. The cage of claim 5 wherein the joint portion is metallic, the proximal sidewall is metallic, and the distal wall comprises a polymer.

9. The cage of claim 2 wherein the joint portion further comprises a flange wherein the flange attaches to the ball and the proximal sidewall.

10. The cage of claim 9 wherein the ball is substantially spherical.

11. The cage of claim 9 wherein the flange is adapted for breakage by manual force.

12. The cage of claim 9 wherein the joint portion is substantially formed within a recess in the proximal surface of the proximal sidewall.

13. The cage of claim 1 wherein the joint portion comprises a recess formed in the proximal sidewall.

14. The cage of claim 13 wherein the recess further comprises a chamfer extending proximally from substantially hemispherical portion.

15. The cage of claim 13 wherein the recess further includes an attachment portion extending distally from the substantially hemispherical portion.

16. The cage of claim 15 wherein the attachment portion is a threaded portion.

17. The cage of claim 13 wherein the recess further includes a channel extending distally from the substantially hemispherical portion.

18. The cage of claim 13 wherein the recess has a substantially hemispherical portion.

19. An assembly comprising:
i) intervertebral fusion cage, comprising:
a) an anterior wall, a posterior wall, a proximal sidewall and a distal sidewall, the sidewalls connecting the anterior wall and the posterior wall to form a throughhole,
b) an upper surface and a lower surface, each surface adapted for gripping a respective adjacent vertebral endplate,
ii) a first joint portion extending from the proximal sidewall and adapted to provide polyaxial movement of the cage, and
iii) an inserter having a proximal handle and a distal end portion, wherein the distal end portion comprises a second joint portion,
wherein the first and second joint portion form a polyaxial joint.

20. The assembly of claim 19 wherein the first joint portion of the cage comprises a ball having a portion of a sphere.

21. The assembly of claim 19 wherein the first joint portion comprises a recess formed in the proximal sidewall and having a substantially hemispherical portion.

22. The assembly of claim 19 wherein the first joint portion of the cage comprises a removable ball comprising mating features that mate with the protrusions in a drive belt inside the inserter, such that the belt can be driven along its guide and rotated about the long axis of the inserter from the instrument's proximal end resulting in the ability to generate and lock infinite polyaxial angles of the implant.

23. A method comprising the steps of:
a) providing the assembly of claim 19,
b) inserting the cage of the assembly into a disc space.
